(19) United States
(12) Patent Application Publication
(10) Pub. No.: US 2025/0195849 A1
(43) Pub. Date: Jun. 19, 2025

(54) BALLOON CATHETER AND METHOD OF PRODUCING BALLOON CATHETER

(71) Applicant: GOODMAN CO., LTD., Aichi (JP)

(72) Inventors: Mitsumasa Okamoto, Aichi (JP); Shoma Kondo, Aichi (JP); Yuta Nakamura, Aichi (JP); Shuhei Yamamoto, Aichi (JP)

(73) Assignee: GOODMAN CO., LTD., Aichi (JP)

(21) Appl. No.: 19/068,499

(22) Filed: Mar. 3, 2025

Related U.S. Application Data
(63) Continuation of application No. PCT/JP2023/039483, filed on Nov. 1, 2023.

(30) Foreign Application Priority Data
Nov. 30, 2022 (JP) 2022-192303

(51) Int. Cl. A61M 25/10

(52) U.S. Cl. CPC A61M 25/1002; A61M 25/1029; A61M 2025/1086; A61M 2207/10

(57) ABSTRACT

A balloon of a balloon catheter includes a straight tube portion in a cylindrical shape with a diameter to be maximum when the balloon is inflated. A linear protrusion protrudes from a surface of the straight tube portion and linearly extends along the surface of the straight tube portion. A rough surface region is formed in each side surface of the linear protrusion. The rough surface region has surface roughness greater than surface roughness of the surface of the straight tube portion.

### CROSS-REFERENCE TO RELATED APPLICATION

The present application is a continuation of International Patent Application No. PCT/JP2023/039483 filed on Nov. 1, 2023, and claims priority to Japanese Patent Application No. 2022-192303 filed on Nov. 30, 2022, the entire contents of which are incorporated herein by reference.

### TECHNICAL FIELD

The present disclosure relates to a balloon catheter and a method of producing a balloon catheter.

### BACKGROUND

A balloon catheter has been used in percutaneous transluminal angioplasty (PTA), percutaneous transluminal coronary angioplasty (PTCA) and other surgeries. The balloon catheter includes an inflatable and deflatable balloon at a tip-end portion of the balloon catheter. The balloon may be deflated and inserted into a section of the blood vessel that is narrowed or obstructed by a lesion and then inflated to dilate the section. The balloon includes a straight tube portion and two tapered portions. The straight tube portion has a cylindrical shape with a diameter that is to be maximum when the balloon is inflated. The tapered portions are disposed on a base-end side and a tip-end side of the straight tube portion. Each tapered portion has a diameter that decreases as a distance from the straight tube portion increases.

A balloon catheter may include a linear protrusion that protrudes from a surface of a straight tube portion and extends along the surface of the straight tube portion (an example may be found in International Publication WO2020/255923). When the balloon is inflated in a section with a lesion, the linear protrusions dig into the lesion to create incisions in the lesion. The section with the lesion can be easily dilated with the incisions as a trigger of the dilation.

When the balloon is inflated to dilate the section with the lesion in the body, the lesion may push back the balloon and thus balloon may slip in the axial direction. If that happens, the balloon may not be able to properly dilate the section with the lesion.

To reduce the slipping of the balloon during the inflation of the balloon, surface roughness of the surface of the straight tube portion of the balloon may be increased. However, this may increase a sliding resistance of the balloon during the insertion of the balloon into a tube such as a blood vessel and may decrease ease in passing of the balloon through the tube.

### SUMMARY

The present disclosure has been made in view of the above circumstances. A main objective of the present disclosure is to provide a balloon catheter that includes a balloon that is less likely to slip during inflation of the balloon although ease in passing of the balloon through a tube is less likely to decrease, and to provide a method of producing the balloon catheter.

A balloon catheter in an embodiment described herein includes a balloon and a linear protrusion. The balloon is inflatable and deflatable. The balloon includes a straight tube portion that is in a cylindrical shape with a diameter to be maximum when the balloon is inflated. The linear protrusion protrudes from a surface of the straight tube portion and extends along the surface of the straight tube portion. The linear protrusion has a surface that includes at least one rough surface region with surface roughness greater than surface roughness of the surface of the straight tube portion.

According to the configuration, when the balloon is inflated and the linear protrusion is pressed against a subject in a target section to be dilated such as a section with a lesion, the rough surface region of the linear protrusion contacts the subject in the target section to be dilated. Therefore, the balloon is less likely to slip during inflation of the balloon.

Because an area of the surface of the straight tube portion that has the less surface roughness is significantly larger than an area of the surface of the linear protrusion, a sliding resistance of the balloon during insertion of the balloon into a tube such as a blood vessel can be reduced. According to the configuration, the balloon is less likely to slip during the inflation of the balloon while the ease in passing of the balloon through the tube is less likely to decrease.

The balloon of the balloon catheter may include a wing that may be formed when the balloon is deflated and may be folded along an outer periphery of the balloon. Further, the linear protrusion may extend in an axial direction of the balloon. The linear protrusion may be covered by the wing from an outer side when the balloon is deflated.

Because the linear protrusion may be covered by the wing from the outer side when the balloon is deflated, the at least one rough surface region of the linear protrusion may be less likely to slide on a tube wall during insertion of the deflated balloon into the tube. Therefore, the ease in passing of the balloon may be further less likely to decrease.

The at least one rough surface region of the balloon of the balloon catheter may be formed only in an intermediate portion of the linear protrusion with respect to a longitudinal direction of the linear protrusion, or the surface roughness of the at least one rough surface region may decrease toward an end of the linear protrusion with respect to the longitudinal direction.

The linear protrusion may not be entirely covered by the wing. For example, an end portion of the linear protrusion may stick out of the wing.

According to the former configuration, because the at least one rough surface region may not be formed in the end portion of the linear protrusion with respect to the longitudinal direction, the sliding resistance may be less likely to increase even if the end portion of the linear protrusion may stick out of the wing and may slide on the tube wall during the insertion of the balloon that may be deflated into a tube. Therefore, the ease in passing of the balloon may be further less likely to decrease. According to the latter configuration, because the surface roughness of the at least one rough surface region in the end portion may be less, similar effects as the effects achieved by the former configuration can be achieved.

The at least one rough surface region of the balloon catheter may include rough surface regions in the surface of the linear protrusion. The rough surface regions may be arranged such that a density of the rough surface regions may increase toward a peak of the linear protrusion.

When the balloon is inflated until the linear protrusion is pressed against a subject in a target section to be dilated such as a lesion, the peak of the linear protrusion may mainly contact the subject. According to the configuration in which the density of the rough surface regions may increase toward the peak of the linear protrusion, the slipping of the balloon during the inflation of the balloon may be properly reduced. Because the density of the rough surface regions may decrease toward a base of the linear protrusion (opposite from the peak), the sliding resistance of the balloon during the insertion of the balloon into the tube may be reduced in comparison to a configuration in which the density of the rough surface regions may be high for the entire area of the surface of the linear protrusion. Therefore, the slipping of the balloon may be properly reduced although the ease in passing of the balloon may be further less likely to decrease.

The surface roughness of the at least one rough surface region of the balloon catheter may increase toward the peak of the linear protrusion.

According to the configuration, the slipping of the balloon during the inflation of the balloon may be properly reduced. Further, the sliding resistance of the balloon during the insertion of the balloon into the tube may be reduced in comparison to the configuration in which the surface roughness may be greater for the entire area of the surface of the linear protrusion. Therefore, the slipping of the balloon may be properly reduced although the ease in passing of the balloon may be further less likely to decrease.

The balloon may include two tapered portions that may sandwich the straight tube portion. The tapered portions may decrease in diameter toward farther sides from the straight tube portion. The tapered portions may include surfaces with surface roughness greater than the surface roughness of the surface of the straight tube portion. The surface roughness of the surfaces of the tapered portions may be less than the surface roughness of the at least one rough surface region.

During dilation of the section with the lesion using the tapered portions of the balloon, the tapered portions may contact the lesion at an angle and thus the lesion may push back the tapered portions, which may cause slipping of the balloon in the axial direction. According to the configuration described above, the surfaces of the tapered portion contact the lesion, which may reduce the slipping of the tapered portions in the axial direction and the slipping of the balloon in the axial direction. Because the surface roughness of the surfaces of the tapered portions may be less than the surface roughness of the rough surface region of the linear protrusion, the effects described above can be achieved without a reduction in ease in passing of the balloon into the tube.

In a method of producing the balloon catheter, a parison in a tubular shape is a base material of the balloon. The parison includes a protrusion on an outer periphery of the parison. The parison is to be formed into the linear protrusion. A mold that is used for producing the balloon includes an internal space for forming the balloon. The internal space includes a space section for forming the straight tube portion. The mold includes an inner wall surface that defines the internal space and includes a wall surface section that defines the space section. The mold includes a groove in the wall surface section for forming the linear protrusion. The mold includes an inner surface that defines the groove and includes a rough surface section with surface roughness greater than surface roughness of the wall surface section. The method includes an expanding process of expanding the parison in the internal space until the outer periphery of the parison is pressed against the inner wall surface and the parison is pushed into the groove to form the linear protrusion. The at least one rough surface region is formed in the surface of the linear protrusion by pushing the protrusion into the groove and pressing the protrusion against the rough surface section in the expanding process.

According to the method, the rough surface region is formed during the expansion of the parison. Therefore, a downstream process to form the rough surface region, such as scraping of the surface of the linear protrusion after the balloon with the linear protrusion is formed through the expansion of the parison, is not required. That is, the balloon catheter in the first aspect is relatively easily produced.

The expanding process in the method of producing the balloon catheter may include a first expanding step and the second expanding step. The first expanding step may include pressurizing an inside of the parison at a first pressure to expand the parison until the protrusion may be inserted into the groove. The second expanding step may include pressurizing the inside of the parison at a second pressure that may be higher than the first pressure to push the protrusion into the groove until the protrusion may be pressed against the rough surface section.

Through the first expanding step, the parison may be relatively slowly expanded and thus the protrusion of the parison may be properly inserted into the groove. Then, the inside of the parison may be pressurized at the second pressure that may be higher than the first pressure to push the protrusion into the groove until the protrusion may be pressed against the rough surface section (the second expanding step). Through the second expanding step, the protrusion may be pushed into the groove and thus the protrusion may be pressed against the rough surface section. Therefore, the rough surface region may be properly formed in the surface of the linear protrusion.

The first expanding step may include heating the parison at a first temperature and the second expanding step may include heating the parison at a second temperature that may be higher than the first temperature.

Because the parison may be heated at the temperature that may be higher than the temperature in the first expanding step (the second temperature), the protrusion of the parison that may be softened may be pushed into the groove. Therefore, the protrusion may be more easily pressed against the rough surface section and thus the rough surface region may be properly formed in the linear protrusion.

In the method, the protrusion may have a height greater than a depth of the groove.

Because the height of the protrusion of the parison may be greater than the depth of the groove, the protrusion may be properly inserted into the groove until the protrusion may reach a bottom of the groove and thus the protrusion may be properly pressed against the rough surface section. Therefore, the rough surface region may be properly formed even in an area adjacent to the peak of the linear protrusion. With such a rough surface region, the slipping of the balloon during the inflation of the balloon may be properly reduced.

In the method, the groove may have a cross section in a V-shape and the protrusion may have a cross section in an inverted V-shape. The inner surface that may define the groove may include two side surfaces that may form the V-shape. The protrusion may include a surface that may include two side surfaces that may form the inverted V-shape. The side surfaces of the protrusion may form an angle that may be greater than an angle formed by the side surfaces that may define the groove.

According to the configuration, the protrusion may deform along the side surfaces that may define the groove during the pushing of the protrusion into the groove and thus the protrusion may be pressed against the side surfaces that may define the groove (or rather the rough surface section). That is, the configuration described above may be preferable for the formation of the rough surface region in the linear protrusion in the inverted V-shape.

In the method, an angle of each of the side surfaces relative to a width direction of the groove may be greater at an opening section of the groove in comparison to a bottom section of the groove.

In the configuration in which the cross section of the groove may be in the V-shape and the cross section of the protrusion may be in the inverted V-shape, the protrusion may slide on the side surfaces that may define the groove after the protrusion may be inserted into the groove, which may cause a problem such as improper formation of the rough surface region in the side surfaces of the linear protrusion. According to the configuration described earlier, the protrusion inserted into the groove may be less likely to slide along the side surfaces that define the groove. Therefore, the problem due to the sliding of the protrusion may be less likely to occur.

In the method, the groove may have a width that may decrease toward a bottom of the groove. The inner surface that may define the groove may include two side surfaces adjacent to each other and joined to each other at a bottom of the groove. The expanding process may include pushing the protrusion into the groove until the protrusion may contact the side surfaces that may define the groove and then the bottom of the groove.

According to the method, the protrusion may be properly pressed against the entire area of the side surfaces that may define the groove (or rather the rough surface section) and thus the rough surface region may be properly formed in the linear protrusion.

### BRIEF DESCRIPTION OF THE DRAWINGS

FIG. 1 is a schematic overall side view illustrating a configuration of a balloon catheter.

FIG. 2A is a side view illustrating a configuration of a balloon that is inflated and therearound.

FIG. 2B is a cross-sectional view cut along line A-A in FIG. 2A.

FIG. 2C is a magnified cross-sectional view illustrating a linear protrusion that is illustrated in FIG. 2B.

FIG. 3 is a side view of the balloon that is inflated and therearound with a lengthwise cross-sectional view of the balloon and an outer tube.

FIG. 4A is a side view of the balloon that is deflated and therearound.

FIG. 4B is a cross-sectional view cut along line B-B in FIG. 4A.

FIG. 5A is a cross-sectional view illustrating a configuration of a mold.

FIG. 5B is a cross-sectional view cut along line C-C in FIG. 5A.

FIG. 5C is a magnified cross-sectional view illustrating a groove that is illustrated in FIG. 5B.

FIG. 6 is a perspective view of a parison.

FIG. 7A is a cross-sectional view illustrating the parison that is expanded in an internal space of the mold.

FIG. 7B is a cross-sectional view cut along line D-D in FIG. 7A.

FIGS. 8A to 8C are cross-sectional views illustrating steps through which a protrusion of the parison is pushed into the groove of the mold in an expanding process.

FIG. 9 is a side view illustrating a configuration of a balloon and therearound in another embodiment.

FIG. 10 is a magnified side view of a linear protrusion with rough surface regions in another embodiment.

FIG. 11 is a cross-sectional view of a groove in a mold in another embodiment.

FIG. 12 is a side view illustrating a configuration of a balloon and therearound in another embodiment.

### DETAILED DESCRIPTION OF EMBODIMENTS

An embodiment of the present disclosure will be described with reference to the drawings.

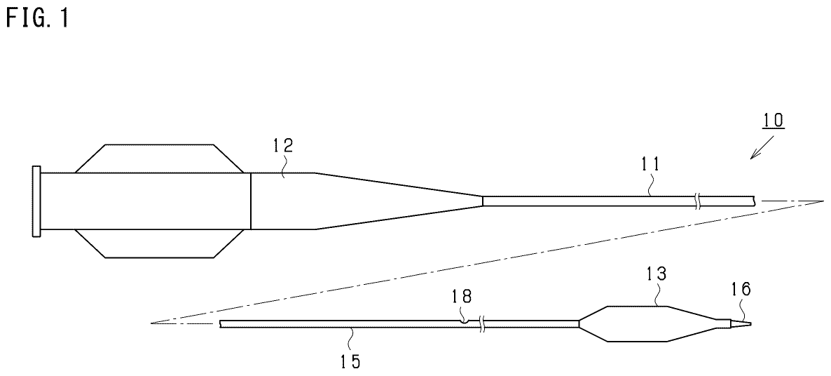

As illustrated in FIG. 1, a balloon catheter 10 includes a catheter tube 11, the hub 12, and a balloon 13. The hub 12 is connected to a base-end portion (a proximal portion) of the catheter tube 11. The balloon 13 is mounted to a tip-end portion (a distal portion) of the catheter tube 11.

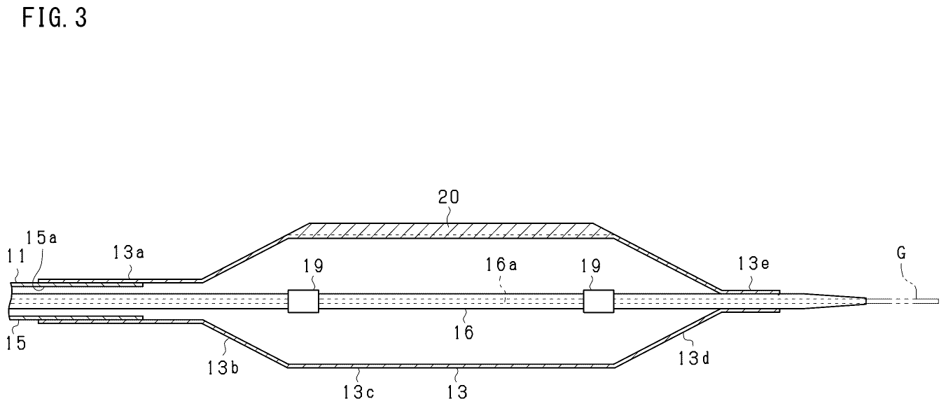

The catheter tube 11 includes an outer tube 15 and an inner tube 16 that is inserted in the outer tube 15. The outer tube 15 is made of a resin material and formed in a tubular shape. The outer tube 15 includes a fluid lumen 15a inside (see FIG. 3). The fluid lumen 15a extends through the outer tube 15 in an axial direction of the outer tube 15 for an entire length. A base-end portion of the outer tube 15 is joined to the hub 12, and the tip-end portion of the outer tube 15 is joined to the balloon 13. The fluid lumen 15a of the outer tube 15 is communicated with an internal space of the hub 12 and an internal space of the balloon 13. For inflation and deflation of the balloon 13, a compressed fluid is passed through the fluid lumen 15a of the outer tube 15.

The outer tube 15 may be composed of multiple tubes that are disposed in a row with respect to the axial direction and joined to one another. One of the multiple tubes on the base-end side may be made of a metal material and another one of the multiple tubes on the tip-end side may be made of a resin material.

The inner tube 16 is made of a resin material and formed in a tubular shape. The inner tube 16 includes a guidewire lumen 16a inside (see FIG. 3). A base-end portion of the inner tube 16 is joined to a middle portion of the outer tube 15 with respect to the axial direction of the outer tube 15. A portion of the inner tube 16 on the tip-end side extends farther than a tip of the outer tube 15 and inserted in an internal space of the balloon 13. A tip-end portion of the inner tube 16 is joined to a tip-end portion of the balloon 13.

The guidewire lumen 16a of the inner tube 16 is provided for passing a guidewire G. A base-end opening of the guidewire lumen 16a is located in the middle of the balloon catheter 10 with respect to the axial direction of the balloon catheter 10. That is, the balloon catheter 10 is classified as an RX-type catheter. A base-end opening 18 of the guidewire lumen 16a may be located at a base-end portion of the balloon catheter 10. This type of the balloon catheter 10 may be classified as an over-the-wire-type catheter.

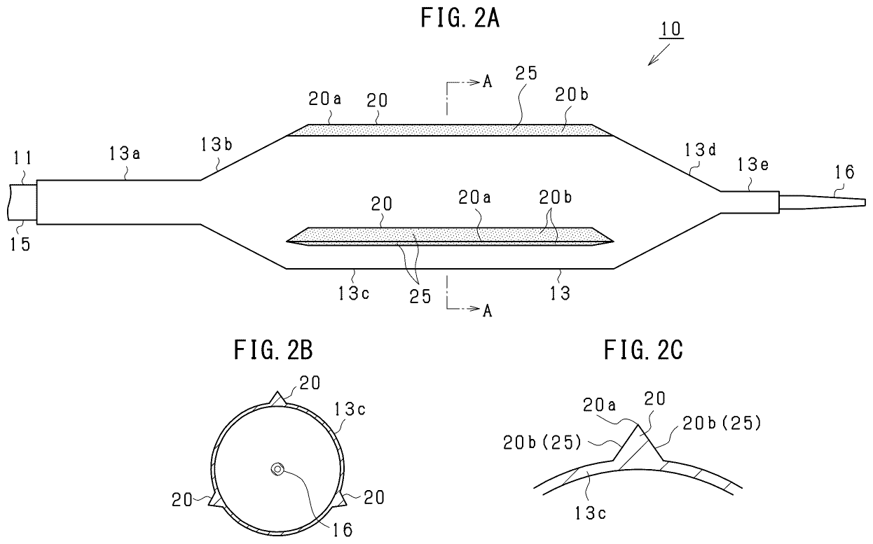

A configuration of the balloon 13 and therearound will be described with reference to FIGS. 2A to 4B.

The balloon 13 is made of a thermoplastic resin material such as polyamide elastomer. As illustrated in FIG. 2A and FIG. 3, the balloon 13 is formed in a cylindrical shape (a tubular shape) with a circular cross section as a whole. Specifically, the balloon 13 includes a base-end leg portion 13a, a base-end tapered portion 13b, a straight tube portion 13c, a tip-end tapered portion 13d, and a tip-end leg portion 13e in this sequence from the base-end side toward the tip-end side.

The base-end leg portion 13a is joined to the tip-end portion of the outer tube 15. The base-end tapered portion 13b increases in diameter from the tip-end of the base-end leg portion 13a toward the tip-end side. That is, the base-end tapered portion 13b is in a tapered shape. The straight tube portion 13c extends from the tip-end side of the base-end tapered portion 13b toward the tip-end side with a constant diameter. That is, the straight tube portion 13c is in a round tubular shape (a cylindrical shape). The straight tube portion 13c has a diameter that is to be maximum when the balloon 13 is inflated. The tip-end tapered portion 13d extends from the tip-end of the straight tube portion 13c toward the tip-end side with a diameter that decreases toward the tip-end side. That is, the tip-end tapered portion 13d is in a tapered shape. The tip-end leg portion 13e is joined to the tip-end portion of the inner tube 16.

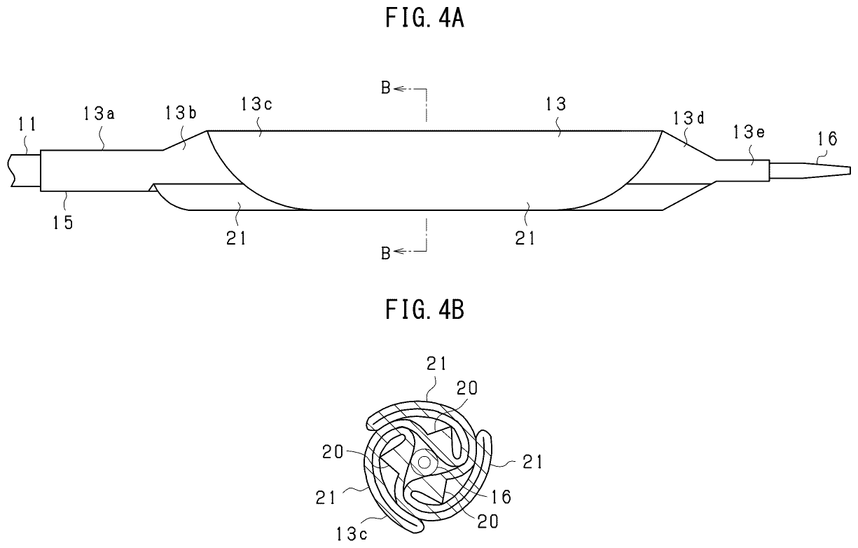

When the compressed fluid is supplied to the internal space of the balloon 13 via the fluid lumen 15a of the outer tube 15, the balloon 13 inflates. When the negative pressure is applied to the fluid lumen 15a of the outer tube 15 and the compressed fluid is discharged from the internal space of the balloon 13, the balloon 13 deflates. As illustrated in FIGS. 4A and 4B, the balloon 13 that is deflated includes multiple (three in this embodiment) wings 21 that appear when the balloon 13 is deflated. The wings 21 are disposed at predetermined intervals (specifically, equal intervals) in the circumferential direction of the balloon 13. Each of the wings 21 extends in the axial direction over the tapered portions 13b, 13d and the straight tube portion 13c. When the balloon 13 is deflated, the wings 21 are folded along the outer periphery of the balloon 13 and wound around the inner tube 16.

Two contrast rings 19 are attached to the inner tube 16 inside the balloon 13. The contrast rings 19 are for improving visibility of the balloon 13 in x-ray images to easily position the balloon 13 to a target section for treatment.

In the balloon catheter 10, linear protrusions 20 protrude from an outer surface of the straight tube portion 13c of the balloon 13 and linearly extend along the outer surface. The linear protrusions 20 are for creating incisions in a lesion during dilation of a section with the lesion by inflating the balloon 13. With the balloon catheter 10, the section with the lesion can be easily dilated by creating incisions in the lesion with the linear protrusions 20 and using the incisions as a trigger of the dilation. That is, the balloon catheter 10 is a balloon catheter having a scoring function.

The linear protrusions 20 protrude from the outer surface of the straight tube portion 13c and extend in the axial direction of the balloon 13 along the outer surface of the straight tube portion 13c. Specifically, the linear protrusions 20 extend in the axial direction for the entire length of the straight tube portion 13c. The linear protrusions 20 are disposed at predetermined intervals (specifically, equal intervals) in the circumferential direction of the balloon 13. In this embodiment, three linear protrusions 20 are disposed. The linear protrusions 20 are integrally formed with the balloon 13.

Each linear protrusion 20 has a widthwise cross section (specifically, a cross section along a direction perpendicular to the longitudinal direction of the linear protrusion 20) in an inverted V-shape that protrudes outward in the radial direction of the balloon 13, specifically, in a triangular shape. A protruding end of the linear protrusion 20 is a peak 20a. The linear protrusion 20 includes two side surfaces 20b that are adjacent to each other and joined to each other at the peak 20a.

The side surfaces 20b of the linear protrusion 20 includes rough surface regions 25 that have surface roughness greater than surface roughness of the surface of the straight tube portion 13c. The linear protrusions 20 include the rough surface regions 25, respectively. In FIG. 2A, the rough surface regions 25 are indicated with dot-hatching patterns. The rough surface regions 25 are present for the entire areas of the side surfaces 20b of the linear protrusion 20 (i.e., the entire surfaces of the linear protrusion 20). The surface roughness of the rough surface regions 25 is constant for the entire areas of the rough surface regions 25. The surface roughness of the rough surface regions 25 of all the linear protrusions 20 are equal. The surface roughness of the surface of the balloon 13 is constant for the entire area of the surface of the balloon 13.

In this disclosure, the expression “surface roughness” means calculated mean roughness Ra defined in the Japanese industrial standards (JIS) B0601:2001. The calculated mean roughness Ra is measured according to JIS B0601:2001 using instrument specified in JIS B0651:2001.

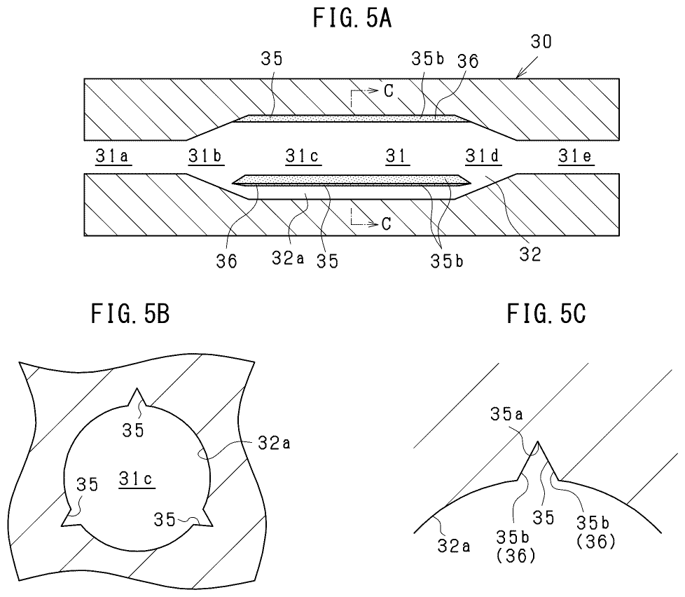

A method of producing the balloon 13 that is described above will be described. The balloon 13 is produced using a mold 30. First, a configuration of the mold 30 will be described with reference to FIGS. 5A to 5C.

As illustrated in FIG. 5A, the mold 30 in a rectangular shape includes an internal space 31 in which the balloon 13 is formed. The internal space 31 is in an elongated shape that extends in the longitudinal direction of the mold 30 and corresponds to the shape of the balloon 13. As illustrated in FIG. 5B, the internal space 31 has a cross section (a cross section along a direction perpendicular to the longitudinal direction of the internal space 31) in a circular shape for an entire length of the internal space 31 in the longitudinal direction.

The internal space 31 includes a space section 31a for forming the base-end leg portion 13a of the balloon 13, a space section 31b for forming the base-end tapered portion 13b, a space section 31c for forming the straight tube portion 13c, a space section 31d for forming the tip-end tapered portion 13d, and space section 31e for forming the tip-end leg portion 13e. The space sections 31a to 31e are in shapes corresponding to the portions 13a to 13e of the balloon 13, respectively.

The mold 30 includes an inner wall surface 32 that defines the internal space 31. The inner wall surface 32 surrounds the internal space 31. The surface roughness of the inner wall surface 32 is constant for the entire area of the inner wall surface 32. Multiple (specifically, three) grooves 35 are formed in an inner wall surface section 32a of the inner wall surface 32 (corresponding to a wall surface section). The inner wall surface section 32a defines the space section 31c of the internal space 31 for the straight tube portion 13c (corresponding to a space section recited in the claims). The grooves 35 extend in the longitudinal direction of the internal space 31. The grooves 35 are at equal intervals in the circumferential direction of the inner wall surface 32.

As illustrated in FIG. 5C, each groove 35 has a widthwise cross section in a valley shape, that is, a V-shape along a direction perpendicular to the longitudinal direction of the groove 35. The cross section of the groove 35 corresponds to the cross section of the linear protrusion 20 (in the inverted V-shape). The groove 35 is defined by two side surfaces 35b that form the valley shape (corresponding to the side surfaces of the inner surface that defines the groove 35). The side surfaces 35b are adjacent to each other and joined to each other at the bottom of the groove 35 (a valley bottom). The groove 35 has a width that decreases toward the bottom 35a of the groove 35.

The side surfaces 35b that define the groove 35 includes rough surface sections 36 with the surface roughness greater than the surface roughness of the inner wall surface 32 (32a). The rough surface sections 36 are present for the entire areas of the side surfaces 35b of each groove 35. The surface roughness of the rough surface sections 36 is constant for the entire areas of the rough surface sections 36. In FIG. 5A, the rough surface section 36 is indicated with dot-hatching patterns.

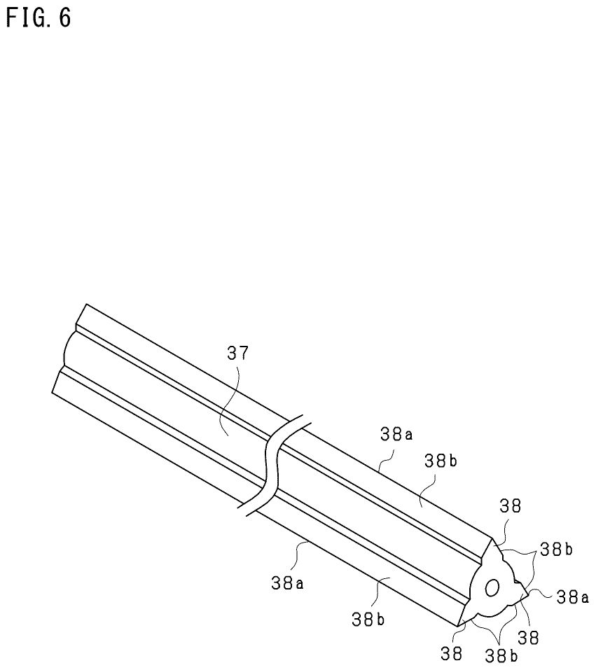

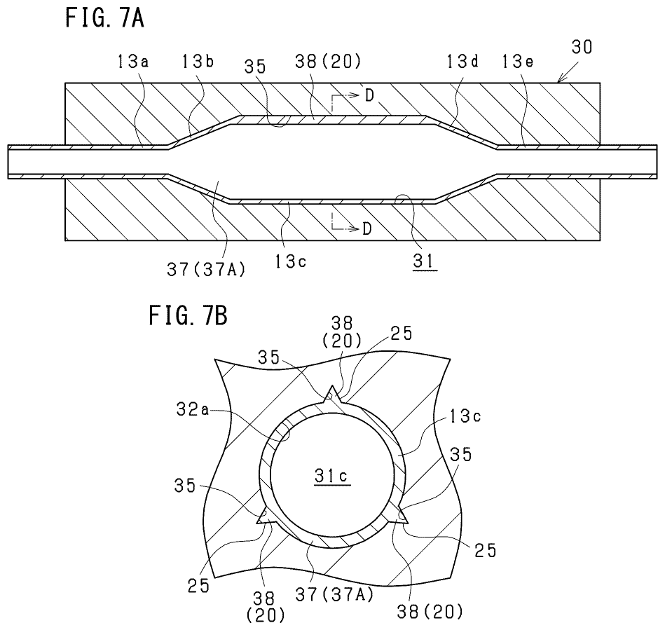

A method of producing the balloon 13 using the mold 30 will be described with reference to FIG. 6 and FIGS. 7A and 7B.

Prior to the production of the balloon 13, a preparing process is performed to prepare the mold 30 and the parison 37 that is a base material of the balloon 13. As illustrated in FIG. 6, the parison 37 is made of a resin material and formed in a cylindrical shape. The parison 37 may be formed by extrusion molding. The parison 37 includes multiple (specifically, three) protrusions 38 that protrude from the outer periphery of the parison 37. The protrusions 38 are to be formed into the linear protrusions 20 of the balloon 13. Each protrusion 38 has a cross section (specifically, a cross section along a direction perpendicular to the longitudinal direction of the parison 37) in an inverted V-shape, which protrudes outward in the radial direction of the parison 37. More specifically, the cross section of the protrusion 38 has a pentagonal shape, or a home base shape. The protrusion 38 extends in the longitudinal direction of the parison 37, more specifically, extends for the entire length in the longitudinal direction of the parison 37. The protrusion 38 includes two side surfaces 38b (corresponding to a surface of the protrusion 38) that form the inverted V-shape. The side surfaces 38b are adjacent to each other and joined to each other at the peak 38a of the protrusion 38.

A placing process is performed to place the parison 37 in the internal space 31 of the mold 30. In this process, the parison 37 is placed in the internal space 31 with the longitudinal direction of the parison 37 aligned with the longitudinal direction of the internal space 31. Therefore, the parison 37 is placed over the space sections 31a to 31e in the internal space 31. Then, an expanding process is performed to expand the parison 37 in the longitudinal direction.

The expanding process is performed to expand the parison 37 in the internal space 31 of the mold 30 until the outer periphery of the parison 37 is pressed against the inner wall surface 32 as illustrated in FIGS. 7A and 7B. In this process, the parison 37 is expanded by introducing a pressurized fluid such as nitrogen into the inside of the parison 37 to pressurize the inside of the parison 37. Through the expanding process, an expanded parison 37A that includes the portions 13a to 13d of the balloon 13 is formed.

In the expanding process, the pressurized fluid is introduced into the inside of the parison 37 using a fluid supply device. The fluid supply device is capable of adjusting the pressure for pressurizing the inside of the parison 37 by adjusting an amount of the pressurized fluid introduced into the inside of the parison 37. In the expanding process, the parison 37 is heated by a heater included in the mold 30 to expand the parison 37. The heater is capable of adjusting the temperature for heating the parison 37.

As illustrated in FIG. 7B, through the expansion of the parison 37, the protrusions 38 of the parison 37 are pushed into the respective grooves 35 and the protrusions 38 are formed into the shapes of the linear protrusions 20 of the balloon 13. Through the expanding process, the linear protrusions 20 are formed on the straight tube portion 13c of the balloon 13. In the expanding process, the rough surface regions 25 are also formed in the side surfaces 20b of the linear protrusions 20 by pushing the protrusions 38 of the parison 37 into the grooves 35 and pressing the protrusions 38 against the side surfaces 35b (the rough surface sections 36) that define the grooves 35.

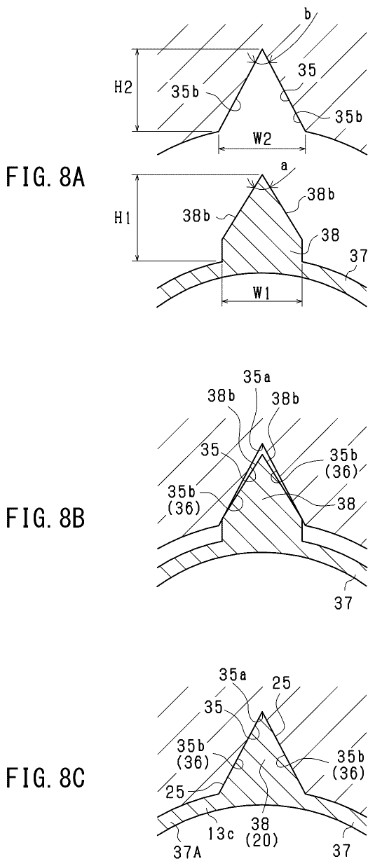

Steps of the expanding process will be described in detail with reference to FIGS. 8A to 8C.

A dimensional relationship between the protrusion 38 of the parison 37 and the groove 35 of the mold 30 will be described with reference to FIG. 8A. As illustrated in FIG. 8A, the width W1 (the maximum width) of the protrusion 38 is less than the width W2 (the maximum width) of the groove 35. The height H1 of the protrusion 38 is greater than the depth H2 of the groove 35. The height H1 is measured from the outer periphery of the parison 37. An angle a that is formed by the side surfaces 38b of each protrusion 38 is greater than an angle b that is formed by the side surfaces 35b of each groove 35.

A flow of the expanding process will be described. A first expanding step in the expanding process illustrated in FIG. 8B is performed to expand the parison 37 until the protrusions 38 of the parison 37 is inserted into the respective grooves 35. In the first expanding step, the parison 37 is pressurized at a first pressure P1, which is relatively low, to expand the parison 37. With the low pressure, the parison 37 relatively slowly expands and thus the protrusions 38 are properly inserted into the grooves 35. Because the width W1 of the protrusions 38 is less than the width W2 of the grooves 35, the protrusions 38 is easily inserted into the grooves 35. In the first expanding step, the parison 37 is heated at a first temperature T1, which is relatively low, to expand the parison 37.

In the first expanding step, the pressure inside the parison 37 (i.e., the first pressure P1) is relatively slowly increased and the heating temperature to heat the parison 37 (i.e., the first temperature T1) is relatively slowly increased. Alternatively, the pressure inside the parison 37 and the heating temperature to heat the parison 37 in the first expanding step may be constant.

A second expanding step illustrated in FIG. 8C is performed to further expand the parison 37 and to push the protrusions 38 into the grooves 35 until the protrusions 38 are pressed against the side surfaces 35b that define the grooves 35 (i.e., the rough surface sections 36). Through the second expanding step, the rough surface regions 25 are formed in the side surfaces 20b of the linear protrusions 20, respectively. In the second expansion step, the parison 37 is expanded by pressurizing the inside of the parison 37 at a second pressure P2 that is higher than the first pressure P1. With the second pressure P2, the protrusions 38 are squeezed into the grooves 35 and thus the protrusions 38 are pressed against the respective side surfaces 35b. Therefore, the rough surface regions 25 are properly formed in the linear protrusions 20.

In the second expanding step, the parison 37 is heated at a second temperature T2 that is higher than the first temperature T1 to expand the parison 37. In this step, the protrusions 38 of the parison 37 are softened and then pushed into the grooves 35. Therefore, the protrusions 38 can be easily pressed against the side surfaces 35b (the rough surface sections 36). That is, this step also enables proper formation of the rough surface regions 25 in the linear protrusions 20.

In the second expanding step, the internal pressure of the parison 37 (i.e., the second pressure T2) is increased more quickly in comparison to the first expanding step, and the temperature to heat the parison 37 (i.e., the second temperature T2) is increased more quickly in comparison to the first expanding step. Alternatively, the internal pressure of the parison 37 and the heating temperature to heat the parison 37 in the second expanding step may be constant.

Because the angle a that is formed by the side surfaces 38a of each protrusion 38 is greater than the angle b that is formed by the side surfaces 35b that define the corresponding groove 35, the protrusions 38 are properly deformed during insertion of the protrusions 38 into the grooves 35 and thus the protrusions 38 are properly pressed against the side surfaces 35b (i.e., the rough surface sections 36). Therefore, the rough surface regions 25 are properly formed in the linear protrusions 20, each of which is in the inverted-V shape.

Because the angle a that is formed by the side surfaces 38b of each protrusion 38 is greater than the angle b that is formed by the side surfaces 35b of each groove 35, the protrusions 38 contact the side surfaces 35b that define the grooves 35 first (see FIG. 8B) and then bottoms 35a of the grooves 35 (see FIG. 8C) during the insertion of the protrusions 38 into the grooves 35. That is, the protrusions 38 are properly pushed until the protrusions 38 reach the bottoms 35a of the grooves 35 while the protrusions 38 deform along the side surfaces 35b that define the grooves 35. Therefore, the protrusions 38 are properly pressed against the entire areas of the side surfaces 35b that define the grooves 35 (and the rough surface sections 36) and thus the rough surface areas 25 are properly formed in the linear protrusions 20.

In the expanding process, portions of the protrusions 38 of the parison 37 that are not inserted into the grooves 35 are pressed against the inner wall surfaces 32 and squashed. Therefore, the linear protrusions 20 are not formed on the portions 13a, 13b, 13d and 13e other than straight tube portion 13c.

After the expansion process, the cutting process is performed to cut off extra end portions of the expanded parison 37A. This completes the formation of the balloon 13 and the production of the balloon 13 is complete.

Then, downstream processes are performed. The downstream processes may include a joining process for joining the balloon 13 to the catheter tube 11 and a joining process for joining the catheter tube 11 to the hub 12. At the completion of the downstream processes, a series of production steps is complete.

A method of using the balloon catheter 10 will be described. A procedure for dilating a section of a blood vessel with a lesion using the balloon catheter 10 will be described.

A guiding catheter is inserted into a sheath introducer that is in the blood vessel and then a tip of the guiding catheter with an opening is inserted into a coronary artery inlet. The guidewire G is then inserted into the guiding catheter and the inserted guidewire G is inserted from the coronary artery inlet to a distal section via the section with the lesion.

Then, the balloon catheter 10 is inserted into the guiding catheter along the guidewire G. After the insertion, pushing force and pulling force are applied until the balloon 13 is placed in the section with the lesion. During the insertion, the balloon 13 remains deflated. When the balloon 13 is deflated, the linear protrusions 20 are covered by the wings 21 from the outer side (see FIG. 4B). Therefore, the rough surface regions 25 of the linear protrusions 20 are less likely to slide on a tube wall during the insertion of the balloon 13. According to the configuration, the ease in passing of the balloon 13 is less likely to decrease even through the linear protrusions 20 include the rough surface regions 25.

After the balloon 13 has reached the section with the lesion, the balloon 13 is inflated. The linear protrusions 20 are pressed against the lesion and incisions (cracks) are created in the lesion by the linear protrusions 20. The incisions may be used as a trigger to break the lesion and to dilate the section with the lesion. Because the rough surface regions 25 of the linear protrusions 20 contact the lesion, the balloon 13 is less likely to slip. According to the configuration, the section with the lesion can be properly dilated at a proper position.

After the dilation of the section with the lesion using the balloon 13 is complete, the balloon 13 is deflated. Then, the balloon catheter 10 with the deflated balloon 13 is removed from the body. This completes a series of steps.

The balloon catheter 10 may be passed through a blood vessel for treatment, for example, treatment for a coronary artery, a femoral artery, a pulmonary artery, or other types of blood vessels. The balloon catheter 10 may be used in treatment of any other types of tubes in living organism other than blood vessels such as ureters and gastrointestinal tract and body cavities.

According to the configuration of the embodiment described in detail above, the following advantageous effects can be achieved.

As described above, the rough surface regions 25 of the linear protrusions 20 contact the lesion when the balloon 13 is inflated and thus the balloon 13 is less likely to slip. Further, the surface roughness of the surface of the straight tube portion 13c of the balloon 13 is less than the surface roughness of the rough surface regions 25 of the linear protrusions 20. The surface of the straight tube portion 13c has a significantly large area in comparison to the surfaces of the linear protrusions 20. Because the surface roughness of the surface of the straight tube portion 13c is less, the sliding resistance of the balloon 13 during passing of the balloon 13 through the blood vessel can be reduced. Therefore, the slipping of the balloon 13 during the inflation of the balloon 13 can be reduced without a reduction in ease in passing of the balloon 13 through the blood vessel.

When the parison 37 is expanded and the protrusions 38 are pushed into the grooves 35 of the mold 30, the protrusions 38 are pressed against the rough surfaces 36 in the grooves 35. This forms the rough surface regions 25 in the side surfaces 20b of the linear protrusions 20. A downstream process such as scraping of the side surfaces 20b of the linear protrusions 20 to form the rough surface regions 25 is not required after the balloon 13 with the linear protrusions 20 is formed by expanding the parison 37. Therefore, the balloon 13 with the linear protrusions 20 that include the rough surface regions 25 is relatively easily produced.

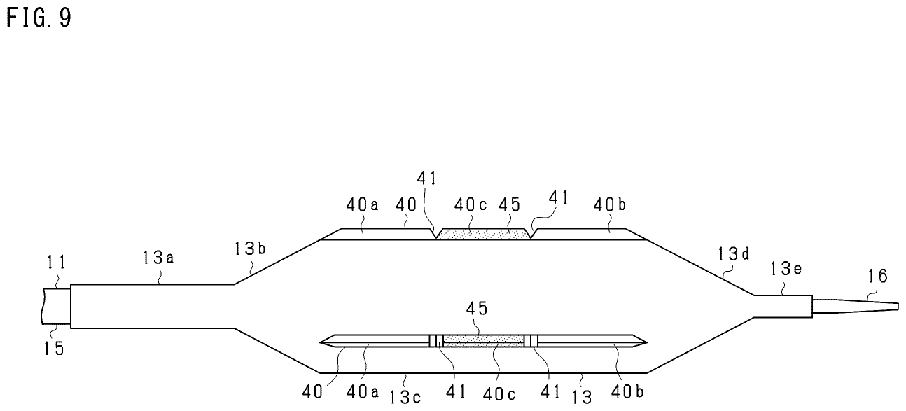

The present disclosure is not limited to the above embodiments and may be implemented as the followings, for example.
(1) In the above embodiment, the rough surface regions 25 are formed in the entire areas of the surfaces of the linear protrusions 20 (specifically, the entire areas of the side surfaces 20b). However, rough surface regions may be formed in sections of the surfaces of the linear protrusions 20. An example is illustrated in FIG. 9. The example illustrated in FIG. 9 includes linear protrusions 40 with notches 41. Multiple (specifically, two) notches 41 are formed in each liner protrusion 40. In each linear protrusion 40, the notches 41 are located to divide the linear protrusion 40 into approximately equal sections (specifically, into thirds).

The linear protrusions 40 include multiple sections 40a to 40c that are separated by the notches 41. The sections 40a to 40c include base-end sections 40a, tip-end sections 40b, and intermediate sections 40c. The base-end sections 40a are located on the base-end side. The tip-end sections 40b are located on the tip-end side. The intermediate sections 40c are located between the base-end sections 40a and the tip-end sections 40b. Rough surface regions 45 are formed in surfaces of the intermediate sections 40c. The surface roughness of the rough surface regions 45 is greater than the surface roughness of the surface of the straight tube portion 13c. The rough surface regions 45 are not formed in the surfaces of the base-end sections 40a and the tip-end sections 40b. That is, only the intermediate sections 40c include the rough surface regions 45 in this embodiment.

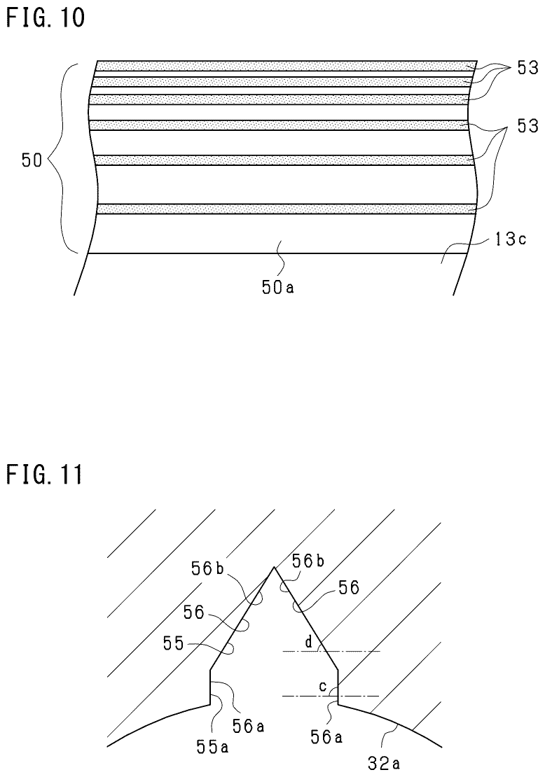

According to the configuration, even if the tip-end sections 40b or the base-end sections 40a of the linear protrusions 40 stick out of the wings 21 and slide on a tube wall during insertion of the deflated balloon 13 into a blood vessel, the sliding resistance is less likely to increase. Therefore, the ease in passing of the balloon 13 through the blood vessel is less likely to decrease.
(2) In the configuration of the above embodiment, the rough surface regions 25 may be configured such that the surface roughness decreases toward the ends of the linear protrusions 20 with respect to the longitudinal direction. For example, the surface roughness of the rough surface regions 25 may be at the maximum at the middle of the linear protrusions 20 with respect to the longitudinal direction. The surface roughness of each rough surface region 25 may decrease from the middle toward one of the ends of the linear protrusions 20 and from the middle toward the other end of the linear protrusions 20 with respect to the longitudinal direction. According to such a configuration, effects similar to the effects described in (1) can be achieved.
(3) Multiple rough surface regions may be formed in each surface of the linear protrusions. An example is illustrated in FIG. 10. The example illustrated in FIG. 10 includes the rough surface regions 53 in each side surface 50a of the linear protrusion 50. Each rough surface region 53 extends in the longitudinal direction of the linear protrusion 50, specifically, extends for an entire length of the linear protrusion 50 in the longitudinal direction. The rough surface regions 53 are arranged at predefined intervals in the side surface 50a of the linear protrusion 50. The rough surface regions 53 are arranged such that intervals between adjacent ones of the rough surface regions 53 decrease toward the peak of the linear protrusion 50. That is, the rough surface regions 53 are arranged such that the density of the rough surface regions 53 increases toward the peak of the linear protrusion 50.

When the balloon 13 is inflated to press the linear protrusion 50 against the lesion, the peak of the linear protrusion 50 primarily contacts the lesion. In the configuration described above, the rough surface regions 53 are arranged such that the density of the rough surface regions 53 increases toward the peak of the linear protrusion 50. Therefore, the balloon 13 is less likely to slip during the inflation of the balloon 13.

The rough surface regions 53 are arranged such that the density of the rough surface regions 53 decreases toward the base of the linear protrusion 50 (i.e., opposite from the peak). Therefore, the sliding resistance of the balloon 13 during the insertion of the balloon 13 into a blood vessel can be reduced in comparison to a configuration in which the rough surface regions 53 are present on each side surface 50a of the linear protrusion 50 with high density for an entire area of the side surface 50a. According to the configuration, the slipping of the balloon 13 is properly reduced without a decrease in ease in passing of the balloon 13.
(4) In the configuration of the above embodiment, the rough surface region 25 may be configured such that the surface roughness increases toward the peak of the linear protrusion 20. According to the configuration, the slipping of the balloon 13 during the inflation of the balloon 13 is properly reduced. In this configuration, the surface roughness of the rough surface region 25 decreases toward the base of the linear protrusion 20. Therefore, the sliding resistance of the balloon 13 during the insertion of the balloon 13 into a blood vessel can be reduced in comparison to a configuration in which the entire rough surface region 25 has greater surface roughness. According to the configuration, similar to the configuration described in (3), the slipping of the balloon 13 is further properly reduced without a decrease in ease in passing of the balloon 13.
(5) The relationship between the dimension of each groove 35 of the mold 30 and the dimension of the corresponding protrusion 38 of the parison 37 is not limited to the relationship in the above embodiment. For example, the height H1 of the protrusion 38 may be less than the depth H2 of the groove 35 or equal to the depth H2 of the groove 35. According to the configuration, the entire protrusion 38 can be properly inserted into the groove 35. The angle a that is formed by the side surfaces 38b of the protrusion 38 may be less than the angle b that is formed by the side surfaces 35b that define the groove 35. According to the configuration, the protrusion 38 of the parison 37 is easily inserted into the groove 35.
(6) The mold 30 may include a groove that has a cross section illustrated in FIG. 11. An example illustrated in FIG. 11 includes a groove 55 that has a cross section in a V-shape similar to that of the embodiment described above. That is, the groove 55 is defined by two side surfaces 56 that form the V-shape. The angle of each side surface 56 relative to the width direction of the groove 55 varies. Specifically, the angle varies between a side surface section 56a of the side surface 56 at an opening section 55a of the groove 55 and a side surface section 56b of the side surface 56 located on a bottom side of the groove 55 than the opening section 55a. More specifically, an angle c of the side surface section 56a relative to the width direction of the groove 55 is greater than an angle d of the side surface section 56b relative to the width direction of the groove 55. For example, the angle c may be 90°.

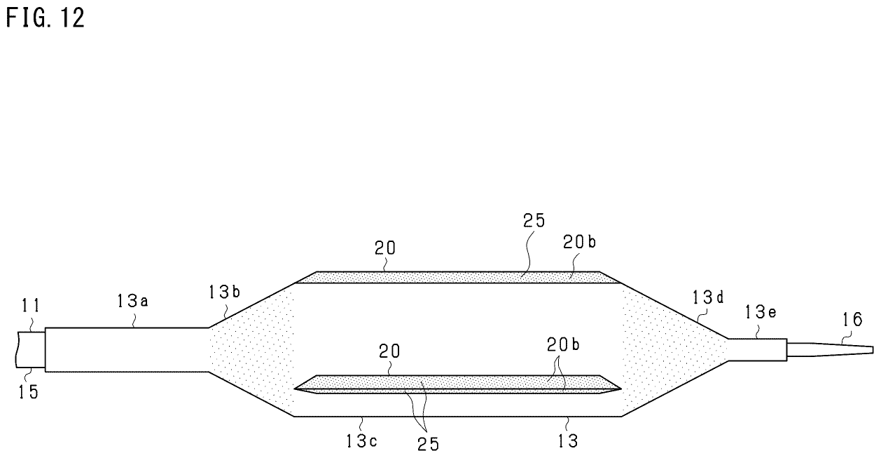

According to the configuration, the protrusion 38 of the parison 37 inserted into the groove 55 in the expanding process is less likely to slide on the side surfaces 56 that define the groove 55. Therefore, problems such as improper formation of the rough surface regions 25 in the side surfaces 20b of the linear protrusion 20 due to the sliding of the protrusion 38 in the groove 55 are less likely to occur.
(7) During the dilation of the section with the lesion by the tapered portions 13b, 13d of the balloon 13, the tapered portions 13b, 13d contact the lesion at an angle and thus the lesion may push back the tapered portions 13b, 13d, which may causes slipping of the balloon 13 in the axial direction. To reduce such a problem, the surfaces of the tapered portions 13b, 13d of the balloon 13 may be configured such that the surface roughness is greater than the surface roughness of the surface of the straight tube portion 13c as illustrated in FIG. 12. According to the configuration, the surfaces of the tapered portions 13b, 13d contact the lesion and thus the tapered portions 13b, 13d are less likely to slip in the axial direction. Further, the balloon 13 is less likely to slip in the axial direction. In FIG. 12, the rough surfaces 13b, 13d are indicated with dot-hatching patterns.

In the example illustrated in FIG. 12, the surface roughness of the surfaces of the tapered portions 13b, 13d is less than the surface roughness of the surfaces of the rough surface regions 25 of the linear protrusions 20. According to the configuration, the effects described earlier can be achieved without a reduction in ease in passing of the balloon 13 through a blood vessel. Alternatively, the surface roughness of only either one of the tapered portions 13b, 13d may be increased.
(8) In the above embodiment, the linear protrusions 20 (first linear protrusions) protrude from the surface of the straight tube portion 13c of the balloon 13. The configuration may further include linear protrusions (second linear protrusions) that protrude from the surface of the tip-end tapered portion 13d. In such a configuration, the surfaces of the second linear protrusions may include rough surface regions with surface roughness greater than the surface roughness of the surface of the straight tube portion 13c. According to the configuration, the balloon 13 is further less likely to slip during the inflation of the balloon 13.

The configuration may also include linear protrusions (third linear protrusions) that protrude from the surface of the base-end tapered portion 13b and include surfaces that include rough surface regions with surface roughness greater than the surface roughness of the surface of the straight tube portion 13c.
(9) In the above embodiment, the rough surface regions 25 are formed in the side surfaces 20b of the linear protrusions 20 in the expanding process to expand the parison 37. However, the method of forming the rough surface regions 25 is not limited to such a method. For example, the balloon 13 with the linear protrusions 20 may be formed and then the side surfaces 20b of the linear protrusions 20 may be scraped to form the rough surface regions 25.

The present disclosure has been described with reference to the embodiments. However, the present disclosure is not limited to the embodiments or the structures. Various modifications and modifications within a scope of equivalents may be within in the technical scope of the present disclosure. Further, various combinations, configurations, and other combinations and configurations including single elements, more or less, may be within the technical scope of the present disclosure.

What is claimed is:

1. A balloon catheter comprising:
a balloon that is inflatable and deflatable and includes a straight tube portion in a cylindrical shape with a diameter to be maximum when the balloon is inflated; and
a linear protrusion that protrudes from a surface of the straight tube portion and extends along the surface of the straight tube portion, wherein the linear protrusion has a surface that includes at least one rough surface region with surface roughness greater than surface roughness of the surface of the straight tube portion.

2. The balloon catheter according to claim 1, wherein
the balloon includes a wing that is formed when the balloon is deflated and folded along an outer periphery of the balloon,
the linear protrusion extends in an axial direction of the balloon, and
the linear protrusion is covered by the wing from an outer side when the balloon is deflated.

3. The balloon catheter according to claim 2, wherein the at least one rough surface region is formed only on an intermediate portion of the linear protrusion with respect to a longitudinal direction of the linear protrusion, or the surface roughness of the at least one rough surface region decreases toward an end of the linear protrusion with respect to the longitudinal direction.

4. The balloon catheter according to claim 1, wherein
the at least one rough surface region includes rough surface regions in the surface of the linear protrusion, and
the rough surface regions are arranged such that a density of the rough surface regions increases toward a peak of the linear protrusion.

5. The balloon catheter according to claim 2, wherein
the at least one rough surface region includes rough surface regions in the surface of the linear protrusion, and
the rough surface regions are arranged such that a density of the rough surface regions increases toward a peak of the linear protrusion.

6. The balloon catheter according to claim 1, wherein the surface roughness of the at least one rough surface region increases toward a peak of the linear protrusion.

7. The balloon catheter according to claim 2, wherein the surface roughness of the at least one rough surface region increases toward a peak of the linear protrusion.

8. The balloon catheter according to claim 1, wherein
the balloon includes two tapered portions that sandwich the straight tube portion,
the tapered portions decrease in diameter toward farther sides from the straight tube portion,
the tapered portions include surfaces with surface roughness greater than the surface roughness of the surface of the straight tube portion, and
the surface roughness of the surfaces of the tapered portions is less than the surface roughness of the at least one rough surface region.

9. The balloon catheter according to claim 2, wherein
the balloon includes two tapered portions that sandwich the straight tube portion,
the tapered portions decrease in diameter toward farther sides from the straight tube portion,
the tapered portions include surfaces with surface roughness greater than the surface roughness of the surface of the straight tube portion, and
the surface roughness of the surfaces of the tapered portions is less than the surface roughness of the at least one rough surface region.

10. A method of producing the balloon catheter according to claim 1, wherein
a parison in a tubular shape is a base material of the balloon and includes a protrusion on an outer periphery of the parison and to be formed into the linear protrusion,
a mold that is used for producing the balloon includes an internal space for forming the balloon,
the internal space includes a space section for forming the straight tube portion,
the mold includes an inner wall surface that defines the internal space and includes a wall surface section that defines the space section,
the mold includes a groove in the wall surface section for forming the linear protrusion, and
the mold includes an inner surface that defines the groove and includes a rough surface section with surface roughness greater than surface roughness of the wall surface section,
the method comprising an expanding process of expanding the parison in the internal space until the outer periphery of the parison is pressed against the inner wall surface and the protrusion is pushed into the groove to form the linear protrusion, wherein the at least one rough surface region is formed in the surface of the linear protrusion by pushing of the protrusion into the groove and pressing of the protrusion against the rough surface section in the expanding process.

11. The method according to claim 10, wherein the expanding process comprises:
a first expanding step of pressurizing an inside of the parison at a first pressure to expand the parison until the protrusion is inserted into the groove; and
a second expanding step of pressurizing the inside of the parison at a second pressure that is higher than the first pressure to push the protrusion into the groove until the protrusion is pressed against the rough surface section.

12. The method according to claim 11, wherein
the first expanding step includes heating the parison at a first temperature, and
the second expanding step includes heating the parison at a second temperature that is higher than the first temperature.

13. The method according to claim 10, wherein the protrusion has a height greater than a depth of the groove.

14. The method according to claim 11, wherein the protrusion has a height greater than a depth of the groove.

15. The method according to claim 10, wherein
the groove has a cross section in a V-shape,
the protrusion has a cross section in an inverted V-shape,
the inner surface that defines the groove includes two side surfaces that form the V-shape,
the protrusion includes a surface that includes two side surfaces that form the inverted V-shape, and
the side surfaces of the protrusion form an angle greater than an angle formed by the side surfaces that define the groove.

16. The method according to claim 11, wherein
the groove has a cross section in a V-shape,
the protrusion has a cross section in an inverted V-shape,
the inner surface that defines the groove includes two side surfaces that form the V-shape,
the protrusion includes a surface that includes two side surfaces that form the inverted V-shape, and
the side surfaces of the protrusion form an angle greater than an angle formed by the side surfaces that define the groove.

17. The method according to claim 15, wherein
an angle of each of the side surfaces relative to a width direction of the groove is greater at an opening section of the groove in comparison to a bottom section of the groove.

18. The method according to claim 16, wherein
an angle of each of the side surfaces relative to a width direction of the groove is greater at an opening section of the groove in comparison to a bottom section of the groove.

19. The method according to claim 10, wherein
the groove has a width that decreases toward a bottom of the groove,
the inner surface that defines the groove includes two side surfaces adjacent to each other and joined to each other at the bottom of the groove, and
the expanding process includes pushing the protrusion into the groove until the protrusion contacts the side surfaces that define the groove and then the bottom of the groove.

20. The method according to claim 11, wherein
the groove has a width that decreases toward a bottom of the groove,
the inner surface that defines the groove includes two side surfaces adjacent to each other and joined to each other at the bottom of the groove, and
the expanding process includes pushing the protrusion into the groove until the protrusion contacts the side surfaces that define the groove and then the bottom of the groove.
